(19) United States
(12) Patent Application Publication
(10) Pub. No.: US 2007/0078332 A1
(43) Pub. Date: Apr. 5, 2007

(54) METHOD OF POSITION LANDMARKING USING A TOUCH SENSITIVE ARRAY

(75) Inventors: Samuel John Wheeldon, Hartland, WI (US); William Todd Peterson, Sussex, WI (US); Jason Lee Philps, Menomonee Falls, WI (US); Lawrence Howard Golding, Milwaukee, WI (US)

Correspondence Address: CANTOR COLBURN, LLP, 55 GRIFFIN ROAD SOUTH, BLOOMFIELD, CT, 06002, US

(73) Assignee: GENERAL ELECTRIC COMPANY, Schenectady, NY (US)

(21) Appl. No.: 11/163,012

(22) Filed: Sep. 30, 2005

(51) Int. Cl. A61B 5/05

(52) U.S. Cl. 600/411

(57) ABSTRACT

A position landmarking system operative with respect to an object includes an operative device including a reference point and configured for movement in a direction relative to the object. The position landmarking system also includes a sensor configured for detecting a stimulus at a location on the sensor and to be disposed relative to the object and corresponding to the direction of movement of the operative device. In response to the stimulus, the reference point of the operative device and the location on the sensor are aligned with each other via movement of the operative device relative to the object. The operative device may be configured for performing an operation relative to a base, the operative device and the base configured for movement relative to each other. The sensor may be configured for communication with the operative device, the base or both.

### BACKGROUND OF THE INVENTION

This invention relates generally to positioning systems and specifically to a landmarking positioning system and method of use thereof.

When prescribing an exam in a magnetic resonance (MR) workflow, one of the first steps is to register the anatomical region that will be imaged to a known or desired location on the anatomical unit. This location on the anatomical unit is moved via the table or platform into alignment with the isocenter of the magnet of the MR imaging device. Once the imaging device is positioned, the imaging exam begins. The process of selecting the desired imaging position is known as landmarking.

Some applications involve the use of laser or halogen alignment lights that project a crosshair onto the object to be imaged. The crosshair is registered to a reference point of the imaging device, such as the isocenter of the magnet of an MR imaging device.

The operator must perform a number of steps to bring the imaging device in line with the desired exam location of a patient or anatomical object. For instance, the operator must turn on the alignment lights, move the region of interest (ROI) to the alignment lights, center the ROI under the crosshairs, press a landmark button to lock the location and press an advance to scan button which takes the landmarked position of the ROI to the isocenter of the magnet. This process limits the workflow of an exam by requiring a minimum of four separate user interactions. There also exists a distinct possibility that the operator will overshoot or undershoot moving the ROI when attempting to align the ROI with the cross hairs. Overshooting or undershooting requires the operator to move the ROI back and forth a number of times to finally align the ROI with the desired location with reference to the imaging device.

There remains a need for a more streamlined landmarking technique with fewer required user interactions that would provide a reliable positioning method for a variety of imaging systems.

### BRIEF DESCRIPTION OF THE INVENTION

In an exemplary embodiment of the invention, a position landmarking system operative with respect to an object includes an operative device including a reference point and configured for movement in a direction relative to the object. The position landmarking system also includes a sensor configured for detecting a stimulus at a location on the sensor and to be disposed relative to the object and corresponding to the direction of movement of the operative device. In response to the stimulus, the reference point of the operative device and the location on the sensor are aligned with each other via movement of the operative device relative to the object.

In another exemplary embodiment, a position landmarking system operative with respect to an object includes a base, an operative device including a reference point and configured for movement in a direction relative to the object. The position landmarking system also includes a sensor configured for detecting a stimulus at a location on the sensor and for communication with the base, the operative device or with both. The base and the operative device are configured for movement in a direction relative to each other and the sensor is disposed to reflect the direction of movement. In response to the stimulus, the reference point of the operative device and the location on the sensor are aligned with each other.

In an exemplary embodiment of a method of position landmarking, the method includes providing a stimulus to a sensor to define a location on the sensor and communicating the location on the sensor to an operative device. The operative device includes a reference point which is aligned with the location on the sensor in response to the stimulus and the communication of the location on the sensor.

### BRIEF DESCRIPTION OF THE DRAWINGS

Referring to the exemplary drawings wherein elements are numbered alike in the several Figures:

FIG. 1 shows a side view of an exemplary embodiment of a position landmarking system according to an embodiment of the invention;

FIG. 2 shows a top view of another exemplary embodiment of a position landmarking system according to an embodiment of the invention;

FIG. 3 shows a side view of another exemplary embodiment of a position landmarking system according to an embodiment of the invention;

FIG. 4 shows a top view of the position landmarking system of FIG. 3 according to an embodiment of the invention;

FIG. 5 shows a side view of another exemplary embodiment of position landmarking system according to an embodiment of the invention; and

FIG. 6 illustrates a laboratory experiment demonstrating an exemplary embodiment of a sensor for a position landmarking system according to an embodiment of the invention.

### DETAILED DESCRIPTION OF THE INVENTION

Disclosed herein in the exemplary embodiments are a system and methodologies that enable a streamlined workflow for a position landmarking technique, with reference to a magnetic resonance (MR) imaging system. While an exemplary system and methodology of positioning an anatomical object relative to a medical MR imaging system is disclosed, it will be appreciated that such disclosure is illustrative only, and it should be understood that the method and system of the disclosed invention may readily be applied to other imaging systems, such as computer tomography (CT) or other scanning systems.

Alternative to “scanning” systems the exemplary embodiments of the positioning system disclosed may also be applicable to other operative devices, such as cameras and manufacturing processing equipment. It should further be noted that the exemplary embodiments include the positioning of anatomical object related to a medical exam, may have application in a variety of imaging fields including, but not limited to industrial evaluation and inspection systems, such as for airport security, and the like that use the imaging technology described above.

In preparing to take an image of an object, users are required to move an object to the proper position relative to an imaging device. The imaging device has a reference point of operation that is aligned with the target location on the object before imaging commences. The user must execute a number of commands, button pushes or switches to move the object, usually on a table or platform, into alignment with the reference point of operation. This “manual” toggling of the object into position often results in the object passing or stopping short of the required position. A sensor in exemplary embodiments of the invention allows the user to choose a location on the sensor relating to a location on the object. The location on the sensor is communicated to the imaging device, whereby the location of the sensor and the reference point of the imaging device are brought into alignment. Minimum interactions of the user are required and the imaging device is reliably brought into alignment with the location on the sensor.

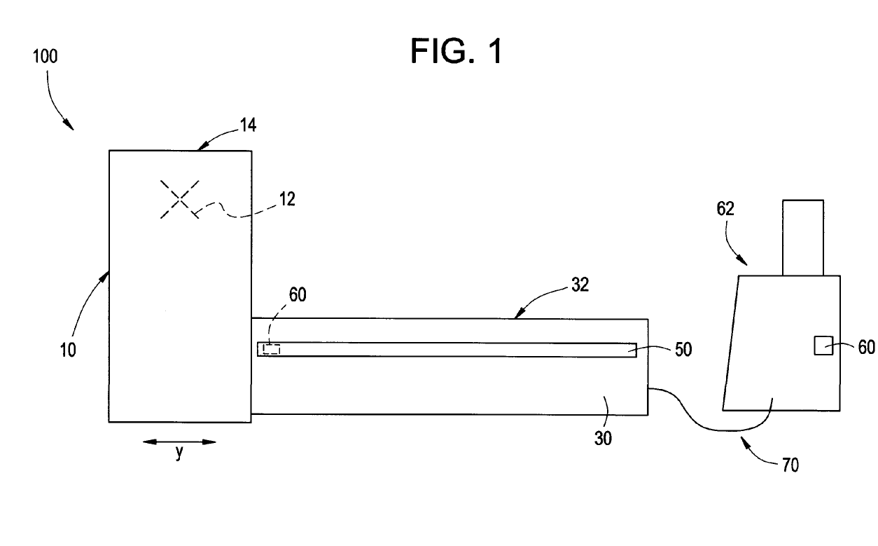

Referring now to FIG. 1, an exemplary embodiment of position landmarking system 100 including an operative device 10, a base 30 and a sensor 50 is depicted. The operative device 10 is configured for performing an operation relative to the base 30. The operative device 10 may include the base 30 or the base 30 may be a separate unit from the operative device 10. The operative device 10 includes a reference point 12 and a moveable portion 14. The sensor 50 may be configured for communication with the base 30, the operative device 10 or both.

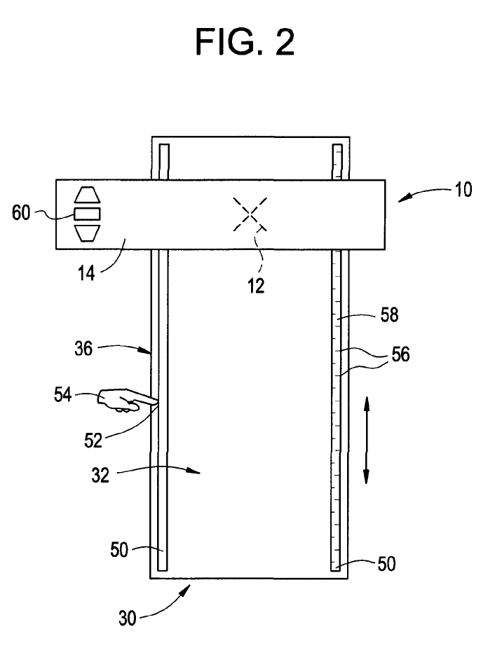

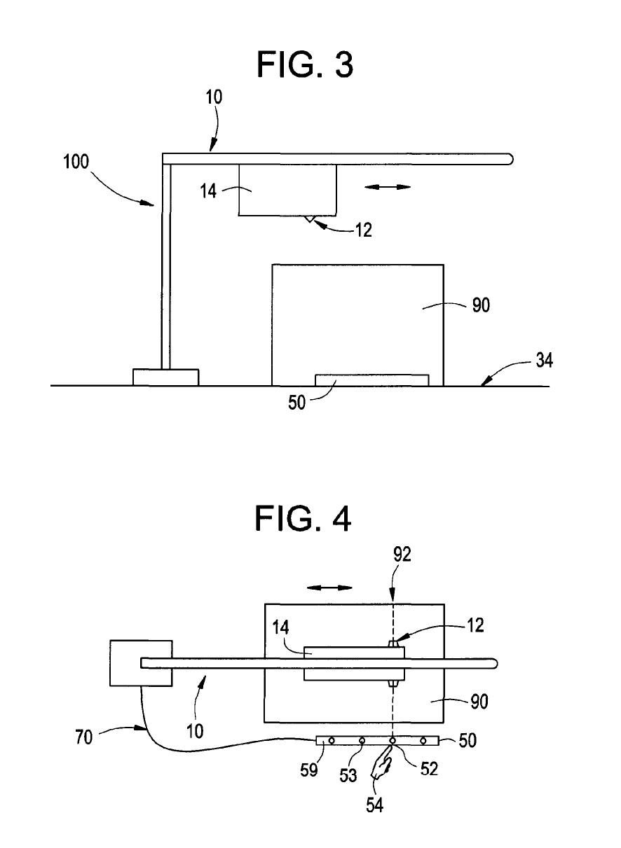

The reference point 12 of the operative device 10 indicates a point of operation for the device with respect to the base 30. For example, the reference point 12 may include an isocenter of a magnet of a MR imaging system or the lens of a camera. The reference point 12 may be an external portion of the operable device 10 as depicted in FIGS. 3-5, or it may be internal to the operative device 10 as illustrated in FIGS. 1 and 2 by the dotted “X”in the moveable portion 14. The operative device 10 may include means to visually indicate on the base 30 where the reference point 12 is positioned on the base 30, such as by a laser or halogen alignment light projecting crosshairs onto the base 30.

As shown in FIGS. 2 and 4, the sensor 50 is configured to detect a stimulus 54 at a location 52 on the sensor 50. In an embodiment, the physical touching of an apparatus (or a person's finger) at location 52 on the sensor 50 is discussed in more detail below. The location 52 on the sensor 50 may correspond with a location on the base 30, such as a side of the base 30 or on the surface 32 of the base 30.

FIG. 1 shows the sensor 50 disposed on the side of the base 30 relative to the surface 32. More than one of the sensor 50 may also be disposed on the base 30. The disposition of the sensor 50 corresponds to the direction of movement of the operative device 10 and the base 30 relative to each other. For example, the sensor 50 in FIG. 1 spans the base 30 lengthwise; substantially parallel to surface 32, in the direction of the arrow “y”. Referring to FIG. 2, the sensor 50 is disposed lengthwise on the surface 32 of the base 30 corresponding to the direction of movement of the base 30 indicated by the arrow “y”. Here, two of the sensor 50 are disposed on the surface 32 of the base 30 proximate to peripheral edges 36 of the surface 32.

When an object 90 (See FIGS. 3 and 4) is placed on the surface 32 of the base 30, the sensor 50 may be considered disposed relative to the object 90. In alternative embodiments, the sensor 50 maybe place on a surface of the operable device 10 instead of the base 30, so long as the disposition of the sensor 50 corresponds to the direction of movement of the operative device 10 and the base 30 relative to each other.

The sensor 50 may be configured to be removably or non-removably disposed on the base 30. For example, the sensor 50 may be a permanent part of the structure of the base 30. To facilitate cleaning, replacement or repair, the sensor 50 may be removable from the base 30. Removably disposing the sensor 50 on the base 30 may also be useful to allow the user to place the sensor 50 in a more advantageous position relative to the object 90 placed on the base 30, such as immediately adjacent to the object 90 or in close proximity thereto.

The operative device 10 and the base 30 may be configured for movement in a direction relative to each other whereby, in response to the stimulus 54, the reference point 12 of the operative device 10 and the location 52 of the stimulus 54 on the sensor 50 are aligned. For example, as illustrated in FIG. 1, the moveable portion 14 of the operative device 10 may move relative to the base 30 in a direction indicated by the arrow. In alternative embodiments, the base 30 may move relative to the operative device 10, as shown in the exemplary embodiment of FIG. 2. Here, the base 30 moves in a direction of the arrow towards the operative device 10 until the location 52 on the sensor 50 is aligned with the reference point 12. Advantageously, minimum interactions of the user with the position landmarking system 100 are required to reliably bring the operative device 10 into operative alignment with the location on the sensor 50. If the location 52 on the sensor 50 is changed or chosen incorrectly, the user may simply re-align the operative device 10 with another chosen location 52 on the sensor 50 by minimum interactions with the position landmarking system 100.

In another exemplary embodiment, the position landmarking system 100 includes a module 60 configured for communication between at least two of the operative device 10, the base 30 and the sensor 50. The module 60 may include a microprocessor, or the like, configured for performing positional or distance calculations based on data from the location 52 on the sensor 50 of the stimulus 54. The calculations may be used to generate instructions causing the operative device 10 or the base 30 to move relative to the other to align the reference point 12 and the location 52 on the sensor 50. For example, the sensor 50 may communicate the location 52 on the sensor 50 to the module 60. The module 60 may then calculate or process the position of the stimulus 54 and communicate the position to the operative device 10. The operative device 10 may then move to align the reference point 12 with the location 52 on the sensor 50.

The operative device 10, the base 30 and/or the sensor 50 may include the module 60. In alternative embodiments, the module 60 may be in included in an external data storage or processing device 62, such as a computer or control unit, related to the operative device 10 or the base 30.

FIGS. 3 and 4 show a side view and a top view, respectively, of another exemplary embodiment of a position landmarking system 100, including the operative device 10, the sensor 50 and an object 90. The operative device 10 is configured to perform an operation relative to the object 90, such as scanning an image or taking a photograph/picture. The operative device 10 includes a reference point 12 and a moveable portion 14.

The operative device 10 may be mobile such that it can be brought to an object 90. For example, the object 90 may be permanently place, such as a fixture or machine in a manufacturing environment. The object 90 may also not be moveable for security or safety reasons, such as a suspect package in an airport or an injured person. Alternatively, the operative device 10 may be fixed, due to its size or installation requirements, for example, and the object 90 is brought to the operative device 10.

Referring to FIGS. 3 and 4, the sensor 50 is disposed relative to the object 90 on surface 34, such as the ground or a floor. The disposition of the sensor 50 corresponds to the direction of movement of the operative device 10 as shown by the arrow. For example, the sensor 50 is disposed substantially parallel to the direction of movement of the operative device 10 in the direction of the arrow. The sensor 50 in FIG. 4 is placed in close proximity to the object 90 such that the sensor 50 spans a target location 92 on the object 90.

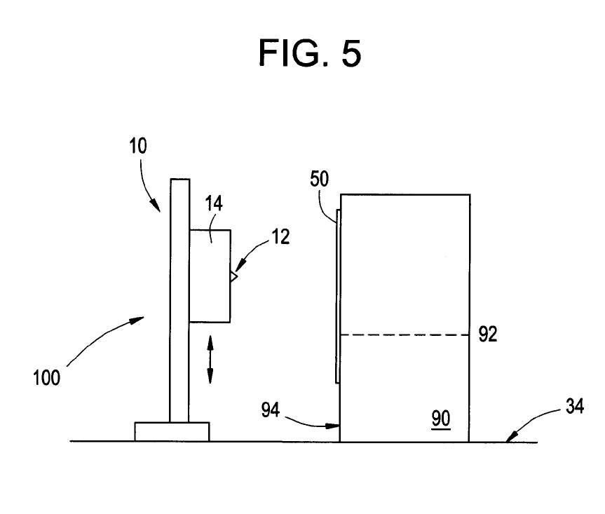

In alternative embodiments, the sensor 50 may be placed on the object 90 itself. For example, FIG. 5 depicts the sensor 50 on a vertical surface 94 of the object 90, spanning the target location 92. Here, the sensor 50 is disposed corresponding to the direction of movement of the operative device 10 relative to the object 90, as shown by the arrow. Essentially, the sensor 50 is configured to be portable, allowing it to be placed on or in proximity to the object 90.

The sensor 50 may be configured as to not interfere with the operation of the operative device 10. For example, if the operative device 10 is an x-ray imaging system, the sensor 50 may be radio-transparent or radio-translucent so as to not significantly interfere with the x-ray imaging or be conspicuous in the generated x-ray image.

Depicting the direction of movement relative to the surfaces of the base 30 and/or the object 90 as substantially parallel is presented for illustration purposes only. In exemplary embodiments, the base 30 and/or the object 90 may include a curved surface. The operative device 10 may move in a substantially linear path or include an arcuate path commensurate with the curved surface. The base 30 may also tilt or change the plane of its surface 32 to position the target location 92 of the object 90 relative to the reference point 12 of the operative device 10. In another exemplary embodiment, the sensor 50 may be flexible to conform to the curved surface of the base 30 or the object 90.

The sensor 50 may include an attachment means that secures the sensor 50 in the position or location to which it is disposed. Where the sensor 50 is removably attached to the base 30, as discussed above, there may be a recess or corresponding attachment means in the base 30 to receive the sensor 50. Where the sensor 50 is placed on the object 90, the attachment means may include something of a temporary adhesive or magnetic pad to secure the sensor 50 on the object 90. The sensor 50 may simply be of substantial mass and size to remain static in the position that it is disposed, such as when it is placed on the ground or floor 34 next to the object 90.

The sensor 50 may include a continuous sensor 58, a discrete sensor 59 or both. The continuous sensor 58 may be configured to allow a stimulus 54 be applied at essentially any location 52 along the continuous sensor 58, whereas the discrete sensor 59 may include a specific point 53 where a stimulus 54 may be applied.

The continuous sensor 58 may include, but is not limited to, a optical light waveguide, a surface acoustical waveguide (SAW), or the like, as well as any combination including at least one of the foregoing. In exemplary embodiments, the continuous sensor 58 may have markings 56, such as the sensor 50 shown on the right peripheral edge 36 of the base 30 in FIG. 2, or be void of any markings 56 such as the sensor 50 shown in FIG. 1 or on the left peripheral side 36 of the base 30 in FIG. 2.

The discrete sensor 59 may include, but is not limited to an array of button switches, a mechanical button array, a wireless tranceiver, a wired tranceiver, or the like, as well as any combination including at least one of the foregoing. For example, FIG. 4 illustrates a sensor 50 with a specific points 53, shown as circles, where a stimulus 54 may be applied. Here, the reference point 12 of the moveable portion 14 of the operative device 10 is aligned with the location 52 on the discrete sensor 59, the location 52 being one of the specific points 53.

The stimulus 54 may include, but is not limited to physical contact, mechanical movement, an electronic signal, or the like, as well as any combination including at least one of the foregoing. The physical contact may be directly from a user's finger or via the user employing an object 90 configured for use with the sensor 50, such as a special wand or pen. The electronic signal may be from a tool or object 90 configured for use with the sensor 50, such as a remote control device with a transceiver or the external data storage device 62 communicating via wired communication means 70 to the position landmarking system 100. For example, the sensor 50 including the optical light waveguide mentioned above, may operate where depressing an area on the sensor 50 reflects light to detect the stimulus 54. Where the surface acoustical waveguide (SAW) is employed, depressing an area on the strip interrupts the SAW. A mechanical button array may operate such that the depressed area on the sensor 50 is read upon the base 30 moving past the enclosure, or the leading edge of the operative device 10, and the depressed button is cleared.

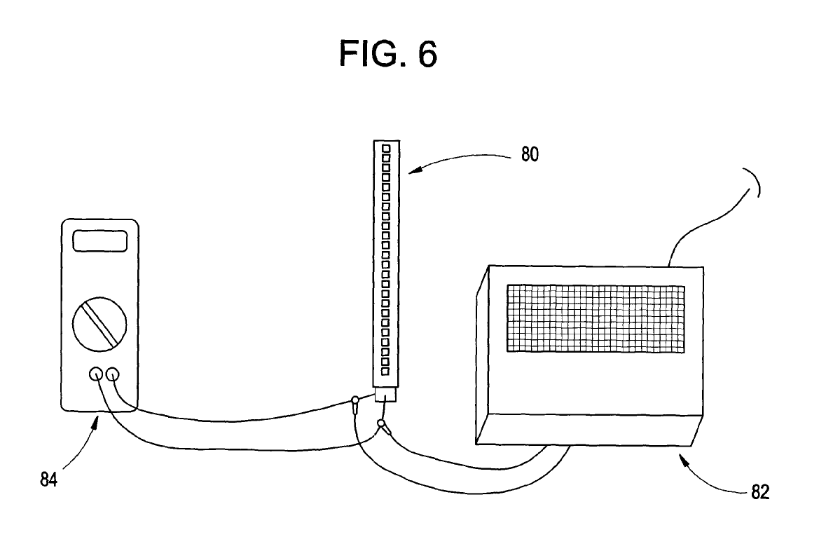

As another exemplary embodiment of the sensor 50, a resistive network of button switches was demonstrated on an exemplary embodiment depicted in FIG. 6. Here, the exemplary embodiment includes a tactile button array 80, a power supply 82 and a voltmeter 84 which were operably connected. A 1-foot, 0.5 inch resolution tactile button array 80 was placed in series with a load resistance of 100 KW. A source voltage of 15 volts was applied and the voltage across the button strip 80 terminals was read with each of the 24 individual buttons pressed using a digital voltmeter 84. The data is detailed in Table 1. below. Each position was easily resolvable into a discrete location without overlap.

| Position Step | Total RL (Ohms) | Vact (measured) |
|---|---|---|
| 1 | 1000 | 0.151 |
| 2 | 2000 | 0.298 |
| 3 | 3000 | 0.443 |
| 4 | 4000 | 0.585 |
| 5 | 5000 | 0.724 |
| 6 | 6000 | 0.8 |
| 7 | 7000 | 0.994 |
| 8 | 8000 | 1.125 |
| 9 | 9000 | 1.253 |
| 10 | 10000 | 1.379 |
| 11 | 11000 | 1.504 |
| 12 | 12000 | 1.626 |
| 13 | 13000 | 1.746 |
| 14 | 14000 | 1.863 |
| 15 | 15000 | 1.97 |
| 16 | 16000 | 2.002 |
| 17 | 17000 | 2.203 |
| 18 | 18000 | 2.313 |
| 19 | 19000 | 2.421 |
| 20 | 20000 | 2.527 |
| 21 | 21000 | 2.631 |
| 22 | 22000 | 2.733 |
| 23 | 23000 | 2.834 |
| 24 | 24000 | 2.933 |
| 25 | 25000 | 3.027 |

The operative device 10 may include, but is not limited to, a computer tomography (CT) scanner, a camera, a measuring instrument, a mechanical tool and the like. It will be appreciated that the exemplary embodiments discussed above may be applicable to a variety of technologies including landmarking information for communication to a system prior to the system performing an operation.

The communication between the operative device 10, the base 30, the sensor 50 and/or the module 60 may be wired or wireless communication. In exemplary embodiments, the wireless communication may include a wireless transceiver in the operative device 10, the base 30, the sensor 50 and/or the module 60. The tranceiver may communicate via radio frequency (RF), infrared (IR), ultrasound (U/S), or the like, as well as any combination including at least one of the foregoing. Wireless RF communication may utilize, for example, 802.11b radio frequency protocol, WI-FI, Bluetooth®, or any other suitable protocol for use with the operative device 10, the base 30, the sensor 50 and/or the module 60.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiments disclosed for carrying out this invention, but that the invention will include all embodiments falling within the scope of the appended claims.

1. A position landmarking system operative with respect to an object, the system comprising:
an operative device configured for movement in a direction relative to the object, said operative device comprising a reference point; and
a sensor configured for detecting a stimulus at a location on said sensor, said sensor configured to be disposed relative to the object;
wherein said operative device and said sensor are configured for operable communication therebetween; and
wherein in response to the stimulus, the reference point of said operative device and the location on said sensor are aligned with each other via movement of said operative device relative to the object.

2. The system of claim 1, wherein said sensor comprises a continuous sensor, a discrete sensor or both.

3. The system of claim 1, wherein said sensor is disposed on the object.

4. The system of claim 3, wherein said sensor is configured to be absent interference with said operative device.

5. The system of claim 1, wherein the communication comprises wired communication, wireless communication or any combination including at least one of the foregoing.

6. A position landmarking system operative with respect to an object, the system comprising:
a base;
an operative device configured for performing an operation with respect to the base, said operative device comprising a reference point; and
a sensor configured for detecting a stimulus at a location on the sensor and for communication with said base, with said operative device or with both;
wherein said base and said operative device are configured for movement in a direction relative to each other, said sensor disposed to reflect the direction of movement;
wherein in response to the stimulus, the reference point of said operative device and the location on the sensor are aligned with each other.

7. The system of claim 6, wherein said operative device includes said base.

8. The system of claim 6, wherein more than one of said sensor is disposed relative to the base.

9. The system of claim 6, wherein said sensor is configured to be disposed on the base.

10. The system of claim 9, wherein said sensor is configured to be removably disposed on the base.

11. The system of claim 9, wherein said sensor is configured to be non-removably disposed on the base.

12. The system of claim 6, further comprising an object disposed on the base, wherein the location on the sensor corresponds to a location on the object.

13. The system of claim 6, the system further comprising a module configured for communication between at least two of said base, said operative device and said sensor.

14. The system of claim 6, wherein said base moves relative to said operative device to align the reference point with the location on the sensor.

15. The system of claim 6, wherein said operative device moves relative to said base to align the reference point with the location on the sensor.

16. A method of position landmarking comprising:
providing a stimulus to a sensor, thereby defining a location on the sensor, and
communicating the location on the sensor to an operative device having a reference point, and in response thereto, aligning the reference point with the location on the sensor.

17. The method of claim 16, wherein said aligning comprises moving the operative device relative to the sensor.

18. The method of claim 16 further comprising disposing the sensor on a base.

19. The method of claim 18, wherein said aligning comprises moving the base relative to the operative device.

20. The method of claim 18, wherein said aligning comprises moving the operative device relative to the base.

21. An object positioning system comprising:
a base to support an object for imaging and translatable in at least one array; and
a sensor proximate the base and configured to detect a stimulus thereto indicating a targeted region of the object to be imaged.

22. The system of claim 21, wherein the sensor is further configured to provide a landmarking signal output to a base controller that when received and processed by the base controller automatically causes the targeted region to be aligned with a reference point of an imaging apparatus that will image the targeted region.

23. The system of claim 22, wherein the base is operably connected to an MR scanner and is translatable fore and aft through a core of the MR scanner, and wherein the landmarking signal output by the sensor causes the base controller to align the targeted region with an optimal imaging volume of the MR scanner.

24. The system of claim 22, wherein the sensor has a length that extends along a surface of the base parallel to a direction of translation of the base.

25. The system of claim 24, wherein the sensor includes a first sensor and a second sensor, the first sensor extending along a first direction of the base and the second sensor extending along a second direction of the base.

26. The system of claim 25, wherein the first sensor and the second sensor each provide a landmarking signal to the base controller that processes the landmarking signals, determine a first position and a second position of the targeted region, and automatically aligns the first and second positions with the reference point of the imaging apparatus.

27. The system of claim 22, wherein the sensor is further configured to receive multiple landmarking stimuli identifying multiple targeted regions of the object to be imaged in a scan session and provide multiple landmarking signals to the base controller that when received and processed causes the multiple targeted regions to be automatically aligned with the reference point during the imaging of each targeted regions during the scan session.

28. The system of claim 21, configured to facilitate operator indication of the targeted region and alignment of the targeted region in a single operator landmarking input.

29. The system of claim 21, wherein the sensor is disposed on an exposed surface of the base.

30. The system of claim 21, wherein the sensor is removably connected to an exposed surface of the base.

31. The system of claim 21, wherein the sensor is removably attached to the object to be imaged.
